(19) United States
(12) Patent Application Publication
(10) Pub. No.: US 2025/0306024 A1
(43) Pub. Date: Oct. 2, 2025

(54) METHOD FOR DETERMINING THE PROGNOSTIC SCORE IN PATIENTS WITH METASTATIC RENAL CARCINOMA

(71) Applicant: UNIVERSITA' DEGLI STUDI DI ROMA "LA SAPIENZA", Roma (IT)

(72) Inventors: Ilaria Grazia Zizzari, Roma (IT); Marianna Nuti, Roma (IT); Aurelia Rughetti, Roma (IT); Chiara Napoletano, Roma (IT); Alessandra Di Filippo, Roma (IT); Paolo Marchetti, Roma (IT); Andrea Botticelli, Roma (IT)

(73) Assignee: UNIVERSITA' DEGLI STUDI DI ROMA "LA SAPIENZA", Roma (IT)

(21) Appl. No.: 18/864,760

(22) PCT Filed: May 31, 2023
(86) PCT No.: PCT/IB2023/055561

§ 371 Date: Nov. 11, 2024

(30) Foreign Application Priority Data
Jun. 3, 2022 (IT) 102022000011795

(51) Int. Cl. G01N 33/574; G16H 10/40; G16H 20/10; G16H 50/30

(52) U.S. Cl. CPC G01N 33/57438; G16H 10/40; G16H 20/10; G16H 50/30; G01N 2333/475; G01N 2333/70517; G01N 2333/70578

(57) ABSTRACT

The present invention relates to a method for rapidly and accurately determining the prognostic score and subsequent division into risk classes, inpatients with metastatic renal carcinoma by acquiring and processing immunological analyses on the patients and combining those immunological analyses with the currently used IMDC classification.

### TECHNICAL BACKGROUND

Renal tumor, which mainly arises from uncontrolled proliferation of the cells that constitute the tubules in which blood filtration occurs, comprises a wide range of histological variants. The most frequent histological variants are the clear cell carcinoma (70-80% of cases), papillary renal carcinoma (10-15% of cases) and chromophobe carcinoma (5% of cases). The renal tumor is a vessel-rich type of tumor with significant angiogenesis. For this reason, one of the main therapeutic strategies currently used, especially in the case of patients with metastatic renal cell carcinoma (mRCC), is the use of molecular targeted drugs, in particular angiogenesis inhibitors, such as tyrosine kinase inhibitors (TKIs), which act by blocking the formation of new vessels capable of supplying oxygen and nutrients to the tumor. Another important therapeutic strategy in the treatment of the metastatic renal tumor is the use of immunotherapy drugs, such as checkpoint inhibitors, which acts by eliminating the “blocking” signals that the tumor creates against the patient's immune system, thus preventing it from recognizing and eliminating the cancer cells. This second approach was found to be particularly effective over others for some patients, depending on their risk classes.

The scientific community agrees that the correct prognosis of mRCC patients, that is, the correct prediction of the course and outcome of a given clinical picture, is an essential step in selecting the type of treatment intended for these patients.

In fact, the use of prognostic factors allows the stratification of the patients themselves according to their disease-related risk of death, provides important information on the evolution of the disease, allows more precise comparisons between clinical trials and facilitates the homogeneous division of the patients in an effort to prevent biases related to patient selection and, consequently, allows the identification of the group for which a given therapeutic treatment has the greatest efficacy.

Currently, the prognostic classification of the mRCC patients is based on integrated models aimed at analyzing, in their totality, clinical, pathological factors and laboratory parameters in order to predict the survival and identify the patients with a high risk of recurrence. The two models that have been most widely used in the clinical practice are the Memorial Sloan Kettering Cancer Center (MSKCC) prognostic system and, more recently, the International Metastatic Renal Cell Carcinoma Database Consortium (IMDC or Heng prognostic system) prognostic system. These two systems have been, and are still currently used, to divide patients into risk classes with the aim of defining precise therapeutic indications for each group.

To date, the most widely used classification is the IMDC classification, which considers six prognostic factors: Karnofsky performance status: <80%; Hemoglobin: below normal range; Corrected calcium: >10 mg/dL; Interval period between diagnosis and treatment: less than 1 year; Absolute neutrophil count: above normal range; Platelet count: above normal range.

Using these prognostic factors, mRCC patients are assigned a score by which they are stratified into three “risk classes” with different prognoses, defined as Good, Intermediate and Poor, (and referred to in the present invention as c-Good, c-Intermediate, c-Poor) corresponding to patients who have a good, intermediate or poor expectation of response to the therapy, respectively.

In particular:
no prognostic factor present: c-Good class
1 or 2 prognostic factors present: c-Intermediate class
3 or more prognostic factors present: c-Poor class.

As mentioned, IMDC criteria are now commonly adopted in clinical practice, although there is clear evidence that the predictive/prognostic role of this model is compromised by the great heterogeneity present among the different risk classes. This observation is particularly relevant for mRCC patients placed in the c-Intermediate class, which may comprise up to 60% of the patients. In fact, this group includes patients having great heterogeneity in IMDC parameters and who actually, although assigned to the same risk class, may have very different prognoses. Furthermore, patients showing only one prognostic factor often have a significantly better prognosis than those showing two negative prognostic factors.

The heterogeneity of the parameters and their possible co-presence deeply influences the response to therapies and the treatment benefit and leads to the clinical result of having patients with different prognoses despite being placed in the same risk class by the IMDC classification criteria. Thus, said classification has not proved to be particularly effective either in grouping patients with the same clinical characteristics or, consequently, capable of significantly increasing the available therapies.

Some studies have proposed integrating the IMDC classification with specific biochemical and clinical parameters, such as, for example, serum levels of C-reactive protein (Kimiharu T et al., Clin Genitourin Cancer 2018), platelet count (Guida A et al., Oncotarget 2020), or else considering the initial site of metastasis (Di Nuzzo V et al., Clin Genitourin Cancer 2018).

In a recent clinical trial, in which mRCC patients were treated with anti-PD1 Nivolumab as mono-therapy, it was found that the simultaneous assessment of clinical and inflammatory parameters contributes to a better prognostic stratification of patients (Rebuzzi S E et al., Ther Adv Med Oncol. 2021).

Fornarini G. et al. also hypothesized that the combination of immune-inflammatory biomarkers, such as the neutrophil-to-lymphocyte ratio (NLR) by platelet number, PD-L1, LDH, is a potentially useful prognostic tool to identify patients who may benefit from immunotherapy alone or alternative therapies (Fornarini G. et al, ESMO Open 2021).

Another recent study showed how a consistent molecular analysis, based on genetic profiles of transcriptional alteration and associated with the clinical response to treatment with anti-VEGF/VEGFR alone or in combination with anti-PD-L1, allowed molecular stratification of mRCC patients by identifying new therapeutic targets which are important for targeted drug development (Motzer R J et al., Cancer Cell 2020). However, while analyzing an important cohort of patients, the method used in this study requires high costs and specific methodologies and responds, to date, to research aspects rather than clinical practice.

Tanaka Nobuyuki et al. (Urologic Oncology: Seminars and Original Investigations, vol. 35, 2016) propose a modified IMDC risk model, in which the neutrophil count, predicted by the standard model as previously described, is replaced by the NLR parameter, neutrophil-to-lymphocyte ratio, in order to improve the predictive ability of the model with respect to the level of survival (Overall Survival, OS). The study demonstrates that, compared with the OS figure, there was an improvement in prediction of 1.7% and 6.2% in the two groups of patients considered (first- and second-line targeted therapy, respectively), with a statistical value of p<0.001.

Chrom Pawel et al. (Int J Clin One, vol. 24, 2019) studied a modified IMDC prognostic model by introducing the systemic immune-inflammation index (SII) based on the neutrophil, lymphocyte and platelet total counts, instead of the neutrophil and platelet counts alone provided by the standard model. The authors find, in general, a higher prognostic accuracy of the new SII-IMDC model than the traditional model with statistical values of p<0.001 with respect to classification into the three risk groups.

Finally, recent studies show that CD137⁺ T-cell populations have a predictive role for response in the TKI and immunotherapy treatments and may therefore be used as biomarkers associated with good prognosis (Zizzari I. et al., Cancers, vol. 12, 2020; Ugolini A. et al., Cancers, vol. 13, 2021, Cirillo A. et al. 2023). This cell subpopulation has never been proposed as a biomarker to be used alone and/or in combination with other parameters for the improvement of the IMDC prognostic score, which is the only criterion still used to date in the classification of patients with metastatic renal cancer.

None of the hypotheses proposed so far has proved to be totally conclusive in identifying an alternative classification method to that used in therapy to date, which is more reliable and easy to apply and which takes into account the immune system as the main target of the new drugs used in clinical practice. Thus, the classification of patients of the Intermediate class remains “imperfect” and incomplete, resulting in a poorly defined and inadequately framed classification.

Thus, there still remains the need to provide simple, reproducible and easily methodologically applicable system able to determine a prognostic classification of patients with metastatic renal carcinoma, which may be more accurate and reliable than the current models used in the clinical practice, in order to get to define a targeted and increasingly effective treatment pathway for the patient.

### OBJECTS OF THE INVENTION

An object of the present invention is to provide a method for determining a new prognostic algorithm in patients with metastatic renal cell carcinoma in a rapid, accurate, reliable and reproducible way that may accurately divide said patients into different risk classes.

Further object of the present invention is the use of the method of the invention for mRCC patients in order to define effective treatment pathways based on the classification obtained.

These and other objects are achieved by the object of the present invention, which aims to establish a method for determining the prognostic score in patients with metastatic renal carcinoma.

### BRIEF DESCRIPTION OF THE FIGURES

FIG. 1: Survival curve, calculated in terms of overall survival (OS) based on IMDC classification alone (graph a), on the top) or Immuno-IMDC classification of the invention (graph b), on the bottom), for the three identified risk categories (Good, Intermediate and Poor).

FIG. 2: Block diagram depicting the Immuno-IMDC prognostic algorithm—of the invention, accompanied by the calculation schemes for scoring the immunological classification (Table 1), the combination of IMDC classification and immunological classification (Table 2) and the immunological score (Table 3).

### DESCRIPTION OF THE INVENTION

The need to increase the effectiveness of stratification of mRCC patients, with respect to the effectiveness of the administered therapy, is, to date, an urgent clinical issue.

The terms “stratify” and “stratification” are identified herein as the action of dividing patients into risk classes, from the lowest to the highest risk, by using the parameters identified by the protocols in use in therapy to date and/or the parameters of the method according to the present invention.

Said stratification is accomplished by the assignment of a score associated with the presence or absence of identified prognostic factors that constitute the basis of the classification as previously described.

The present invention directly addresses the clinical need explicated above, since it identifies a method for determining the prognostic score in patients with metastatic renal carcinoma by acquiring and processing immunological analyses on patients and combining the results of said immunological analyses with IMDC classification. This allows the already established prognostic algorithm to be adjusted with the patient's immunological parameters in relation to new immunotherapy treatment protocols directed precisely at targeting (identifying as a target) the patient's immune system.

As previously pointed out, the stratification of the patients derived from the IMDC classification is referred to herein as c-Good, c-Intermediate, c-Poor.

In particular, object of the present invention is a method which allows identifying the prognostic risk class of mRCC patients, and then stratifying said patients in order to be able to assign the most suitable therapy to each subject based on the expected prognosis, in relation to the fact that the therapies to date used in the treatment of mRCC are based primarily on the activation of the immune system. Said combined approach is in fact precisely constituted by the integration of the current IMDC prognostic classification with the evaluation of two immunological parameters, thus allowing a more accurate assessment of the patient's chances of response to therapy.

According to a particularly preferred aspect of the present invention, the immunological parameters that are taken into consideration, to be used in conjunction with IMDC classification for determining the prognostic score of patients, are two: the serum value of the Vascular Endothelial Growth Factor (VEGF) and the percentage of circulating CD8⁺CD137⁺ T lymphocytes; said parameters are used to define the “Immunological Classification” and the “Immunological Score”.

In particular, the VEGF concentration value in serum is measured from an isolated sample of the patient's serum by one of the methods known to the skilled in the art, preferably by an ELISA test. The percentage value of circulating CD8+CD137⁺ T lymphocytes can be measured from an isolated sample of the patient's blood according to all known techniques, preferably by collecting peripheral blood mononuclear cells (PBMCs) that are subjected to flow cytometry analysis to assess the expression of CD137⁺ on CD8⁺ T lymphocytes. The percentage value of circulating CD8⁺CD137⁺ T lymphocytes is expressed relative to the value of CD3⁺CD137⁺ T lymphocytes.

Therefore, according to the present invention, to determine the patient's prognosis, his or her introduction into one of the three identified risk classes (Good, Intermediate, and Poor) and the subsequent selection of therapy plan, the current IMDC prognostic classification, as per the state of the art, is applied, to which a stratification based on two immunological parameters, namely the serum VEGF concentration value and the percentage of circulating CD8⁺CD137⁺ T lymphocytes, is added.

In particular, said parameters allow the patients to be reclassified from an immunological point of view.

Said parameters, i.e., the serum VEGF concentration value and percentage of circulating CD8⁺CD137⁺ T lymphocytes, were selected following a screening of a number of possible immunological parameters that are identified and reported in the literature as having a correlation with cancer diseases. At the end of this screening, the two reference parameters (serum VEGF and circulating CD8⁺CD137⁺ T lymphocytes) were selected, which, compared with the others, demonstrate to have the greatest statistical contribution in improving the predictive ability of the combined method and, at the same time, were found to be directly related to the action of the therapies used to date in metastatic renal carcinoma (ICI and TKI). In fact, TKIs block the action of the VEGF receptor, thus inhibiting the immunosuppressive and angiogenic action of VEGF; whereas the circulating CD8⁺CD137⁺ T lymphocytes are to date a specific marker of the response to ICI immunotherapy in several solid tumors (Zizzari I. G. et al. 2022; Cirillo A. et al. 2023)

The two selected parameters, despite being known in the literature for their role in tumor differentiation and growth (VEGF) and in activating a specific anti-tumor response (circulating CD8⁺CD137⁺ T lymphocytes), have never been proposed to implement the well-known IMDC prognostic classification, either as single markers or in combination with each other.

For the purpose of assigning the score for the classification, quartiles were identified between the values of serum VEGF concentration and the percentage of circulating CD8⁺CD137⁺ T lymphocytes in the analyzed patients as follows:
25^{th} percentile (first quartile, Q1);
75^{th} percentile (third quartile, Q3);
values falling within the interquartile range (IQR).

In other words, quartiles are position indices that divide an ordered population of data into four groups containing approximately an equal numbers of observations and identify the value below which a given percentage of the distribution falls. The first quartile (Q1), also called the₂₅th percentile, is a value that identifies 25% of the observations below Q1 and excludes the remaining 75%; similarly, the third quartile (Q3), also called the 75⁺ percentile, is the value that identifies 75% of the observations below Q3, excluding the remaining 25%. The interquartile range, IQR, is defined as the difference between the third and first quartiles (Q3-Q1) and is a dispersion index that coincides with the range in which at least 50% of the data are found.

Taking this type of data partitioning into account, dividing the percentage of circulating CD8⁺CD137⁺ T lymphocytes into percentiles allows the patients to be considered as:
T lympho-Good: with CD8⁺CD137+% values ≥75^{th} percentile;
T lympho-Intermediate: with CD8⁺CD137⁺% values falling within the interquartile range, IQR (Q1≤CD8⁺CD137⁺%<Q3, i.e. values between Q1 and Q3);
T lympho-Poor: with CD8⁺CD137⁺% values <25^{th} percentile.

Dividing the serum VEGF concentration value into percentiles allows the patients to be considered as:
VEGF-Good: with serum VEGF values <25^{th} percentile;
VEGF-Intermediate: with serum VEGF values falling within the interquartile range (IQR) (Q1≤VEGF <Q3, i.e. values between Q1 and Q3);
VEGF-Poor: with VEGF values ≥75^{th} percentile.

Combining together, as shown in Table 1, the distribution of patients obtained from the distribution in percentiles of the two immunological parameters just described, the patients were classified according to “Immunological Classification (i)” into: i-Good, i-Intermediate and i-Poor (i=immunological).

TABLE 1
Immunological Classification (i)

| | | Immunological |
|---|---|---|
| VEGF | CD8CD137 | Classification (i) |
| VEGF-Good | T lympho-Good | i-Good |
| (values <25^{th} percentile) | (values ≥75^{th} percentile) | |
| VEGF-Good | T lympho-Intermediate | |
| (values <25^{th} percentile) | (values in the IQR range) | |
| VEGF-Intermediate | T lympho-Good | |
| (values in the IQR range) | (values ≥75^{th} percentile) | |
| VEGF-Good | T lympho-Poor | i-Intermediate |
| (values <25^{th} percentile) | (values <25^{th} percentile) | |
| VEGF-Intermediate | T lympho-Intermediate | |
| (values in the IQR range) | (values in the IQR range) | |
| VEGF-Poor | T lympho-Good | |
| (values ≥75^{th} percentile) | (values ≥75^{th} percentile) | |
| VEGF-Poor | T lympho-Intermediate | i-Poor |
| (values ≥75^{th} percentile) | (values in the IQR range) | |
| VEGF-Poor | T lympho-Poor | |
| (values ≥75^{th} percentile) | (values <25^{th} percentile) | |
| VEGF-Intermediate | T lympho-Poor | |
| (values in the IQR range) | (values <25^{th} percentile) | |

The “Immunological Classification (i)” can then be combined with the parameters of class c-Good and c-Poor patients derived from the IMDC classification according to the scheme in Table 2, allowing a new and better stratification of these two classes of patients and consequently making improvements in the survival curve.

TABLE 2
combination of IMDC classification (c)
and immunological classification (i)

| IMDC (c) | Immunological Classification (i) | Immuno-IMDC |
|---|---|---|
| c-Good | i-Good | Good |
| c-Good | i-Intermediate | |
| c-Good | i-Poor | Intermediate |
| c-Poor | i-Good | |
| c-Poor | i-Intermediate | Poor |
| c-Poor | i-Poor | |

However, the combination of these two classifications still does not allow the optimal discrimination of the c-Intermediate patients.

An “Immunological Score” was then calculated for this class of patients, based on the median value of the percentage of circulating CD8⁺CD137⁺ T lymphocytes and the median value of serum VEGF concentration.

A score of 1 or 0 was assigned for values above or below the median value as follows:
CD8⁺CD137⁺%≥median value, score=1;
CD8⁺CD137⁺%<median value, score=0;
VEGF ≥median value, score=0;
VEGF <median value, score=1.

The arithmetic sum of the individual scores determined an “Immunological Score” for each patient, ranging from 0 to 2. Patients with score 0 were definitely classified as Intermediate, patients with scores of 1 or 2 as Good as schematized in Table 3.

TABLE 3
Immunological Score

| VEGF vs. | CD8⁺CD137⁺ % vs. | | |
|---|---|---|---|
| median value | median value | Score | Immuno-IMDC |
| <score = 1 | <score = 0 | 1 | Good |
| <score = 1 | ≥score = 1 | 2 | |
| ≥score = 0 | ≥score = 1 | 1 | |
| ≥score = 0 | <score = 0 | 0 | Intermediate |

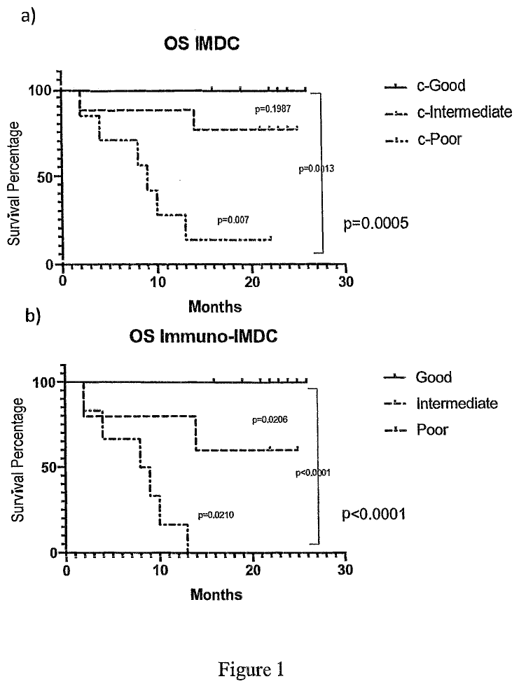

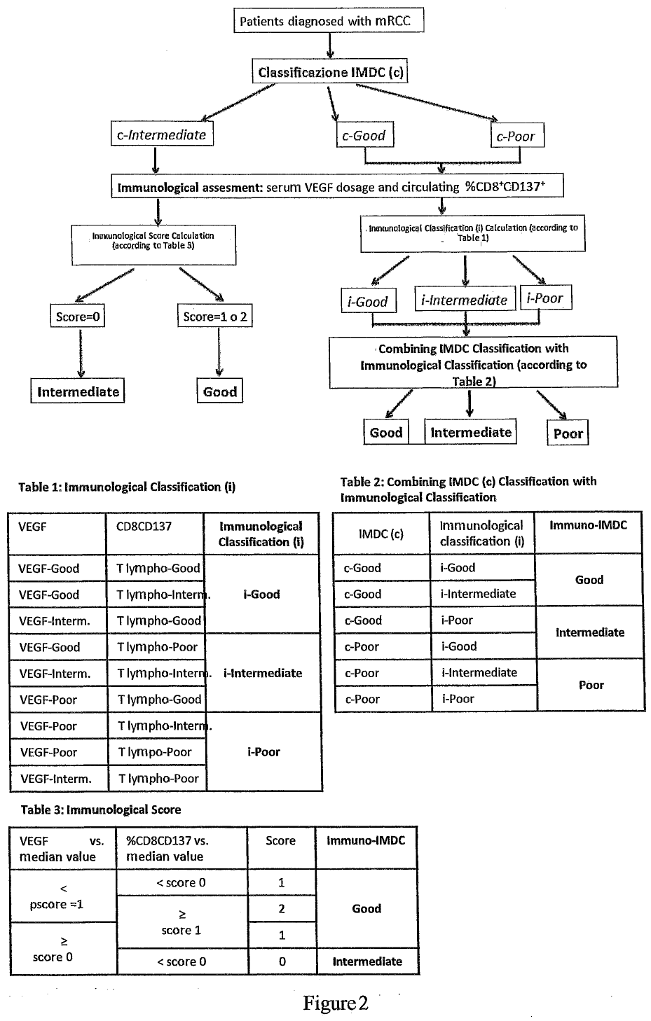

The combination of the IMDC classification and the two immunological parameters just described (Immuno-IMDC combination, schematized in the block diagram in FIG. 2), allowed to obtain a statistically more significant stratification of patients according to the risk classes than the IMDC classification alone (p<0.0001 vs. p=0.0005, respectively), especially for Intermediate patients (p=0.0206 vs. p=0.1987, respectively) (see FIGS. 1a and 1b related to data from the patient population of the experimental section).

Immuno-IMDC has thus allowed the generation of a new prognostic algorithm, according to the scheme shown in FIG. 2, which allows for the reclassification of c-Intermediate (IMDC) patients by combining them to an immunological score and c-Good and c-Poor patients by combining IMDC with the immunological classification (i), significantly improving the prognostic stratification.

In other words, the combination of the two classifications, IMDC and immunological, according to the present invention, a classification called “Immuno-IMDC” has been demonstrated to be able to significantly discriminate patients belonging to the three risk classes.

In particular, experimental data show that the use of IMDC classification carries with it p-values=0.0005 in terms of survival, whereas the combined use with the immunological parameters according to the present invention, Immuno-IMDC analysis, leads to calculated values of p<0.0001 (FIG. 1). In inferential statistics, the p-value is the probability, for a hypothesis assumed to be true, of obtaining results that are equally or less compatible than those observed during the test with the aforementioned hypothesis. In other words, the p-value helps to understand whether the difference between the observed and hypothesized outcomes is due to the randomness introduced by the sampling, or else whether this difference is statistically significant. The closer the p-value is to zero, the more the hypothesis will be verified in reality.

Furthermore, the ability of the method according to the invention to significantly discriminate the survival curves of patients belonging to the Intermediate risk class from the patients in the Good risk class (p-values=0.1987 obtained with IMDC compared with a p-value=0.0206 by using the combined Immuno-IMDC analysis) was experimentally observed; to improve the survival curves between Good and Poor patients (p-values=0.0013 obtained with IMDC compared with a p-value <0.0001 by using the combined Immuno-IMDC analysis) and to maintain the significant difference in survival between Intermediate and Poor patients (p-values=0.007 obtained with IMDC compared with a p-value=0.0210 by using the combined Immuno-IMDC analysis). These values are clearly depicted in FIG. 1, in which the patients' survival curves, calculated in terms of overall survival, are shown according to the IMDC (a), on the top) and Immuno-IMDC (b), on the bottom) classification. The stratification into the different risk classes obtained through the proposed Immuno-IMDC classification, as can be seen from the statistical p-values shown (FIG. 1), appears to be more accurate not only than that obtained through the standard IMDC method, but also than its modifications known to date in the state of the art and previously discussed. Specifically, the proposed Immuno-IMDC stratification stands out for significantly discriminating the OS curves of patients belonging to all three risk classes (Intermediate vs. Good p=0.02; Good vs. Poor p<0.0001; Intermediate vs. Poor p=0.021). From the other modifications known to date in the state of the art (Tanaka N. et al., 2017; Pawel Chrom et al., 2019), a significant overall OS rate is inferred, but the analysis in the various survival curves among different risk classes is not. Furthermore, the overall OS rate calculated by the proposed method is significantly higher than the previously discussed known methods (p<0.0001 vs. p<0.001), demonstrating the superior statistical power of the Immuno-IMDC method described herein.

According to a preferred aspect of the invention, the accurate stratification of patients within the three identified risk classes is achieved thanks to the integration of the IMDC classification with the score obtained by measuring the two identified immunological parameters.

In particular, with regards to the patients who, following the IMDC classification, would be placed within the c-Intermediate category, the use of the immunological score, obtained from the scores derivable from the VEGF and lymphocyte immunological parameters, allows their further subdivision within the Intermediate or Good class if they have a score of 0 or 1-2, respectively, as schematized in Table 3 and FIG. 2. The class of the intermediates is thus redefined more accurately through immunological parameters.

Furthermore, the patients who, according to the IMDC classification, would be placed within the c-Good or c-Poor categories, can be better divided within the three Good, Intermediate, and Poor categories thanks to the integration with the immunological classification (i) according to the diagram in FIG. 2 and Table 2.

The method according to the present invention is thus able to stratify the patients more accurately than the current state of the art from a prognostic point of view, and in particular by taking advantage of the integration between the data obtained thanks to the immunological evaluation constituted by the measurement of the parameter values described above, and those derived from the IMDC classification, so that patients can benefit the most from the therapy administered to them.

As previously set forth, for example, by using only the IMDC classification, in the class of c-Intermediate patients, patients were also included who are instead identified as “Good” thanks to the method of the present invention. Similarly, following the IMDC classification alone, some patients could be placed in the c-Poor or c-Good class but, thanks to what is the subject of the present invention, they will turn out to be able to be defined “Intermediate” instead.

Compared with the alternative methods to the standard IMDC classification found to date in the literature and previously described in the paragraph on the technical background, the method of the present invention does not propose to replace immunological parameters for those already provided in the standard method, but rather complements the subdivision employed in the first instance and obtained by the parameters of the IMDC with the values of serum VEGF concentration and the percentage of circulating CD8⁺CD137⁺ T lymphocytes obtained from the analysis of serum and/or blood samples from patients, as previously described and as schematized in FIG. 2.

The method according to the present invention, therefore, does not relate to the use of a modified IMDC classification, as is the case in the previously cited literature cases, but is characterized by a double level of analysis that allows the patients, already classified in a first step according to the IMDC model, to be re-subdivided in a more predictive way with respect to their prognosis and the treatment they will then need.

By redefining the patients on the basis of clinical and immunologic characteristics, thanks to the method of the present invention that allows to perform a more accurate classification within the subject population, one will then be able to avoid subjecting said subjects to unnecessary high-toxicity treatments and failed therapies, to the benefit of greater therapeutic benefit.

An additional advantage brought by the present invention over the state of the art is that, for its implementation, two easily dosed immunological parameters are used in the blood samples isolated from patients, who will undergo a single evaluation by means of a single, minimal venous sampling, prior to the initiation of therapy, in conjunction with routine clinical tests. Furthermore, the evaluation of said immunological parameters is based on rapid and standardized methods, for example, ELISA assay and flow cytometry assay, which are inexpensive and commonly used in the clinical practice of any hospital facility.

These aspects are also beneficial from an economic point of view, allowing for no significant burden on patients or the National Health Service.

The present invention will now be described, in a non-limiting manner, in the following experimental section.

### EXPERIMENTAL SECTION

Evaluation of the Method of Classifying mRCC Patients into Risk Classes According to the Method of the Invention

23 patients with metastatic renal carcinoma undergoing TKI treatment, classified according to the IMDC score as below:
7 patients classified with good prognosis (c-Good);
9 patients classified with intermediate prognosis (c-Intermediate);
7 patients classified with poor prognosis (c-Poor).

From these patients, PBMCs (peripheral blood mononuclear cells) were collected from isolated blood samples and taken prior to the beginning of the therapy. PBMCs were subjected to flow cytometry analysis to assess the expression of CD137⁺ on T lymphocytes, particularly CD8⁺ lymphocytes.

For the patient population considered, the values corresponding to the quartiles, as previously identified, for this parameter are:
T lympho-Good: with CD8⁺CD137⁺% values ≥2.620% (≥75^{th} percentile);
T lympho-Intermediate: with CD8⁺CD137⁺% values between 0.8 and 2.620% (0.8%≤CD8⁺CD137⁺<2.620%; thus falling within the interquartile range, IQR);
T lympho-Poor: with CD8⁺CD137⁺% values <0.8% (<25^{th} percentile).

Whereas the median value of the percentage of circulating CD8⁺CD137⁺ T lymphocytes is 1%, determining:
CD8⁺CD137⁺%≥1% (median value), score=1;
CD8⁺CD137⁺%<1% (median value), score=0.

At the same time, the serum from the patients was collected and used as an isolated sample to assess the serum VEGF concentration by ELISA assay (Human VEGF Quantikine ELISA Kit, R&D System, cat.no. DVE00).

For the patient population considered, the values corresponding to the quartiles, as previously identified, for this parameter are:
VEGF-Good: with serum VEGF values <192.6 pg/mL (<25⁺ percentile);
VEGF-Intermediate: with serum VEGF values between 192.6 and 472.5 pg/mL (192.6 pg/mL≤VEGF <472.5 pg/mL, thus falling within the interquartile range, IQR);
VEGF-Poor: with VEGF values ≥472.5 pg/mL (≥75^{th} percentile).

Whereas the median value of the serum VEGF concentration is 334 μg/mL, determining:
VEGF ≥334 μg/mL (median value), score=0;
VEGF <334 μg/mL (median value), score.=1.

Based on the clinical classification (IMDC), the overall survival (OS) of the patients was calculated by Kaplan Meyers analysis (p=0.0005). This classification allows c-Good patients to be discriminated well from c-Poor patients, but the survival curve between c-Intermediate and c-Good patients is not statistically significant (p=0.1987). This critical issue has been overcome through the present invention, i.e.:
by calculating the immunological score for c-Intermediate patients;
by combining the IMDC classification with the immunological classification for c-Poor and c-Good patients.

The final stratification resulting from the results of the method according to the present invention overcomes the critical issues found, by improving the final survival curve between Good and Intermediate from a p-value=0.1987 to a statistically significant p-value=0.0206.

1. A method for dividing into risk classes called Good, Intermediate and Poor, mRCC patients according to the IMDC model, wherein said model is supplemented with the results from the measurement in isolated serum and/or blood samples of two immunological parameters which are the serum VEGF concentration values and the percentage of circulating CD8⁺CD137⁺ T lymphocytes of said patients, resulting in the Immuno-IMDC classification.

2. The method according to claim 1, wherein if said serum VEGF values in patients classified c-Intermediate according to the IMDC classification are greater than or equal to the median value, they imply the assignment of a score equal to 0 and if they are lower than the median value, they imply the assignment of a score equal to 1.

3. The method according to claim 1, wherein if said percentages values of CD8⁺CD137⁺ T lymphocytes in patients classified c-Intermediate according to the IMDC classification, are greater than or equal to the median value, they imply the assignment of a score equal to 1, if they lower than the median value they imply the assignment of a score equal to 0.

4. The method according to claim 2, a patient classified c-Intermediate according to the IMDC classification is placed within the Immuno-IMDC classification as:
a. Good in case he scored 1 or 2 from the arithmetic sum of the scores derived from the serum VEGF values and the percentage values of CD8⁺CD137⁺ T lymphocytes;
b. Intermediate in case he scored 0 from the arithmetic sum of the scores derived from the serum VEGF values and the percentage values of CD8⁺CD137⁺ T lymphocytes.

5. The method according to claim 1, wherein said serum VEGF values, in patients classified c-Good or c-Poor according to the IMDC classification, allow patients with serum VEGF values <25^{th} percentile to be classified VEGF-Good; allow patients with serum VEGF values within the interquartile range between Q1 and Q3 i.e., Q1≤VEGF<Q3, to be classified VEGF-Intermediate; allow patients with VEGF values ≥the 75^{th} percentile to be classified VEGF-Poor.

6. The method according to claim 1, wherein said percentage values of CD8⁺CD137⁺ T lymphocytes, in patients classified c-Good or c-Poor according to the IMDC classification, allow patients with CD8⁺CD137⁺% values ≥the 75^{th} percentile to be classified T lympho-Good; allow patients with CD8⁺CD137⁺ values within the interquartile range between Q1 and Q3, i.e., Q1≤CD8⁺CD137⁺<Q3, to be classified T lympho-Intermediate; allow patients with CD8⁺CD137⁺ values <the 25^{th} percentile to be classified T lympho-Poor.

7. The method according to claim 5, a patient classified c-Good or c-Poor according to the IMMDC classification is placed within the immunological classification (i) as:
a. i-Good in case he has been classified, depending on the serum VEGF values and percentage values of CD8⁺CD137⁺ T lymphocytes, VEGF-Good and T-lympho-Good or VEGF-Good and T lympho-Intermediate or VEGF-Intermediate and T lympho-Good, respectively;
b. i-Intermediate in case he has been classified, depending on the serum VEGF values and percentages values of CD8⁺CD137⁺ T lymphocytes, VEGF-Good and T lympho-Poor or VEGF-Intermediate and T-lympho-Intermediate or VEGF-Poor and T lympho-Poor, respectively.

8. The method according to claim 7, wherein a patient classified c-Good or c-Poor according to the IMDC classification is placed within the Immuno-IMDC classification as:
a. Good in case he has been classified, depending on the IMDC model and immunological classification (i) c-Good and i-Good or c-Good and i-Intermediate, respectively;
b Intermediate in case he has been classified, depending on the IMDC model and immunological classification (i) c-Good and i-Poor or c-Poor and i-Good, respectively;
c. Poor in case he has been classified, depending on the IMDC model and immunological classification (i) c-Poor and i-Intermediate or c-Poor and i-Poor, respectively.

9. The method according to claim 1 for defining the treatment course followed by the mRCC patient.
